(19) United States
(12) Patent Application Publication
(10) Pub. No.: US 2007/0073380 A1
(43) Pub. Date: Mar. 29, 2007

(54) Longitudinally expanding, rotating & contracting shaped memory superelastic stent

(76) Inventors: Frank B. Vazquez, Key Biscayne, FL (US); Teresa Luhn Vazquez, Key Biscayne, FL (US)

Correspondence Address: JOHN H. FARO;SUITE 1100, 44 W. FLAGLER STREET, MIAMI, FL, 33130, US

(21) Appl. No.: 11/313,143

(22) Filed: Dec. 19, 2005

Related U.S. Application Data
(60) Provisional application No. 60/637,628, filed on Dec. 20, 2004.

(51) Int. Cl. A61F 2/06

(52) U.S. Cl. 623/1.15

(57) ABSTRACT

An intralumenal tubular assembly comprising two elements of essentially equal length, bonded together preferably by coaxially inserting a rectangular wire into a rectangular thin walled tube, of shape memory superelastic material, helically wound, exhibiting a hysteresis loop in phase transformation and superelastic loading and unloading, following different paths. This composite stent assembly, upon deployment, in an occluded vessel, provides very light continuous contact with the vessel wall, enhanced by the plurality of turns with multiple peaks and valleys to prevent damage to the endothelial cells which secrete several substances that regulate the flexibility and clot formation of the vessels. The composite stent assembly can be deployed in either a compacted or extended configuration, prior to undergoing phase transformation, and relative linear and rotational movement within the occluded vessel.

### CROSS REFERENCE TO RELATED APPLICATION

This application claims priority to Provisional Application Ser. No. 60/637,628, filed Dec. 20, 2005.

### BACKGROUND OF THE INVENTION

1. Field of the Invention

This invention relates to a medical device. More specifically this invention relates to a intralumenal stent of composite construction, which is composed of two elements of a shaped memory metal of essentially equal length; and, a method for use of an intralumenal stent in the treatment of coronary artery disease. In the method of this invention the composite stent is deployed within the diseased vessel; and, as a result of relative movement of the stent, vis-à-vis the clogged vessel, gently displaces plaque by a wiping and twisting action. Such relative movement within the obstructed vessel is accomplished by taking advantage of the transition temperatures of the alloys used in manufacture of each discrete element of the composite stent. Accordingly, the obstructed vessel is not subject to trauma commonly associated with balloon angioplasty, and consequently the likelihood of restenosis is minimized.

2. Description of the Prior Art

In the early 1960's, the inventor was affiliated with the U.S. Naval Ordinance Laboratory, White Oak, Md., where he obtained the first samples of Nickel Titanium Alloy wire when it was in the development stages—they named it NITINOL. At that time, he developed a circuit breaker using Nitinol, instead of Bimetal, based on the shape memory characteristic of Nitinol. Shortly thereafter, in Jan. 23, 1976, the inventor designed a Nitinol wire coil to be delivered in a chilled catheter to arteries in the heart, expanding at body temperature. This coiled wire was, thus, when deployed, in the form of an intralumenal stent. This concept was disclosed, in confidence, to a medical device manufacturer in 1976, who unfortunately was disinterested in the development of this concept. The inventor did not have the resources to pursue this concept independently.

Since 1976, the use of expandable endoprosthesis devices, generally called stents, which are adapted to be implanted into a patient's body lumen, such as a blood vessel, to maintain the patency thereof, have become both a widely accepted and well-known. These devices are recognized to be useful in the treatment of atherosclerotic stenosis in blood vessels. Stents are generally tubular shaped devices, are fabricated from stainless steel, which function to hold open a segment of a blood vessel or other anatomical lumen. They are particularly suitable for use to support and hold back a dissected arterial lining which can occlude the fluid passageway there through.

Further details of prior art stents can be found in the patent literature, specifically, U.S. Pat. No. 3,868,956 (to Alfidi et al.); U.S. Pat. No. 4,512,338 (to Balko et al.); U.S. Pat. No. 4,553,545 (to Maass et al.); U.S. Pat. No. 4,733,665 (to Palmaz); U.S. Pat No. 4,762,128 (to Rosenbluth); U.S. Pat. No. 4,800,882 (to Gianturco); U.S. Pat. No. 4,856,516 (to Hillstead); and U.S. Pat. No. 4,886,062 (to Wiktor); U.S. Pat. No. 5,421,955 (to Lau), and U.S. Pat. No. 5,514,154 (to Lau), which are hereby incorporated herein in their entirety by reference thereto.

Various means have been described to deliver and implant stents. One method frequently described for delivering a stent to a desired intralumenal location includes mounting an expandable stent on an expandable member, such as a balloon, provided on the distal end of an intravascular catheter, advancing the catheter to the desired location within the patient's body lumen, inflating the balloon on the catheter to expand the stent into a permanent expanded condition and then deflating the balloon and removing the catheter.

Alternatively, stents prepared from shaped memory metals, such as Nitinol, can be deployed within an obstruction of a vessel, and expanded without aid of a balloon, and thereby enlarge the previously obstructed lumen.

Lastly, in more extreme cases of vessel blockage, the obstruction (e.g. plaque) can first be dislodged by scraping the deposit from the blood vessel wall, prior to the deployment of the stent. In this method, the use an embolic protection device, of the type disclosed in U.S. Pat. No. 6,702,834 (to Boylan) is generally mandated.

Where the intralumenal wall of the obstructed vessel is subjected trauma (e.g. balloon angioplasty, scraping, etc.), the endothelial response is predictive—restenosis. In order to minimize restenosis, stents have been coated with certain drugs designed to retard an endothelial response to such trauma. These so-called drug coated stents have met with substantial success, notwithstanding, the difficulties encountered in quality control and in their manufacture. Moreover, the exposure of a patient to such drugs is not with risk or adverse reaction.

Accordingly, there continues to exist a need for improvement in both stent design, and the methods for deployment thereof. More specifically, it is both desirable, and medical prudent, to minimize the exposure of an obstructed vessel to trauma, because such trauma introduces complexities into the treatment of disease, which are best avoided. Moreover, the use of powerful drugs to counter the body's natural response to such trauma, is not without finite risk, which is also best avoided. Thus, there continues to be a need for alternatives to traumatic intervention in the treatment of diseases involving obstructive blockages, with medical devices and methods, which does not rely introduction of powerful drugs into the body.

### OBJECTS OF THE INVENTION

It is the object of this invention to remedy the above as well as related deficiencies in the prior art.

More specifically, it is the object of this invention to provide a stent of composite construction, wherein the deployment and subsequent relative movement thereof within an obstructed area of a vessel, both reduces obstructive deposits, and anchors the stent within the lumen of an obstructed vessel, while at the same time minimizes trauma to the endothelial tissue of the obstructed vessel.

It is another object of this invention to provide a stent of composite construction wherein the relative movement of the stent, within an obstructed area of a vessel, is in response to preprogrammed changes in the memory metal characteristics of the stent.

It is still yet another object of this invention to provide a stent of composite construction wherein the relative movement of the stent within an obstructed area of a vessel displaces plaque from the intralumenal wall of the vessel in the obstructed area of the vessel, while at the same time minimizing trauma to the endothelial tissue of the obstructed vessel.

It is an additional object of this invention to provide a method for deployment of a stent within an obstructed area of a vessel.

### SUMMARY OF THE INVENTION

The above and related objects are achieved by providing a stent comprising a pair of discrete elements, which function in conjunction with each other; and, which by virtue of the unique design of such elements, maximizes the prevention of restenosis and undesirable scarring of the walls of the vessel. More specifically, the discrete elements of this stent, upon deployment, extend and rotate within a lumen of an occluded blood vessel, in response to induced temperature changes. The relative linear and rotational movement of the stent, within the lumen of an occluded blood vessel, creates gentle lengthwise and rotating wise wiping or sweeping action, in a manner that is designed to both displace plaque and prevent blood clot formation.

The preferred configuration of the composite stent of this invention comprises a coaxial structure, having two elements, bonded together and helically wound. The composite stent of this invention is made of a shape memory superelastic alloy, such as nickel-titanium alloy, also known as Nitinol, Flexon, Teenee, Memorite, Tinel and Titanium Nickel. The dual element stent of this invention utilizes the unique characteristics of the memory metal to impart the non-traumatic deployment, and effective scaffolding properties, to this composite stent structure.

These superelastic properties of the above nickel titanium alloys, such as Nitinol, are fully utilized in this composite stent, to impart preprogrammed characteristics, and thereby, in the environment of contemplated use, cause it to longitudinally contract, expand and rotate, while keeping a constant outside diameter of the stent in a gentle steady contact with the lumen walls, while at the same time resisting stiffer forces in the opposite direction. More specifically, the unique characteristics of Nitinol which, unlike conventional materials that follow the same linear path in their changes, exhibits a hysteresis loop showing different paths in loading and unloading and stress and strain associated with the Nitinol alloy's superelasticity.

Nitinol also shows a hysteresis in the shape memory property of transformation from the Martensite phase to the Austenite phase, and vice versa. After the dual element is manufactured and assembled it can be constrained in the closed or compact position for insertion into the catheter. This catheter is then guided by conventional means to the obstructed segment in the lumen, where it is deployed by releasing it from the catheter until the lumen is filled, and expansion stopped.

As graphically illustrated in FIG. 5, the computer visualization of the deployment of the composite stents of this invention are preprogrammed to exert gentle pressure against the vessel wall in a controlled manner, as represented by the unloading arrows. A very light total contact pressure of the stent assembly is attained while at the same time, it is highly resistant to forces tending to close the lumen. The total contact pressure must be very low to avoid damage to the vessel wall. Notwithstanding, due to the extensive area of contact of the undulating design, the composite remains secure in the site and in shape desired. In the first embodiment of this invention (FIG. 1) the assembly is deployed in the wound contracted shape; and, in the second embodiment of this invention (FIG. 3), in the unwound and rotated shape.

In the preferred embodiments of this invention, the composite stent assembly comprises two elements of equal length, bonded together preferably by coaxially inserting a rectangular wire into a rectangular thin walled tube, of shape memory superelastic material, helically wound exhibiting a hysteresis loop in phase transformation and superelastic loading and unloading, following different paths, providing very light continuous contact with the vessel wall, enhanced by the plurality of turns, with multiple peaks and valleys, to prevent damage to the endothelial cells which secrete several substances that regulate the flexibility and clot formation of the vessels.

This composite stent assembly is also relatively compliant in the linear dimension and relatively stiff in the radial dimension. Upon induction heating of this composite stent assembly, is changes along the linear dimension concurrent with a rotating action. These combined movements, gently sweep and wipe the occluding materials from the vessel wall. These combined movement of the stent can be induced repeatedly, or periodically, without surgical intervention or re-catherization.

### BRIEF DESCRIPTION OF THE DRAWINGS

FIG. 1 depicts a composite stent of this invention, with one element of the composite stent in the Austenite phase, at body temperature, bonded to the second element in the Martensite phase. In this embodiment of the invention, the composite/assembly is in the wound contracted or short shape.

FIG. 2 depicts the composite stent of FIG. 1 wherein the assembly has expanded and rotated by the pull of the shape programmed for the second element in the Austenite phase, at higher than body temperature.

FIG. 3 depicts a composite stent of this invention with the composite/assembly expanded unwound and rotated at body temperature, wherein the second element has been programmed for that shape at body temperature while the first element has been programmed to the short wound shape at higher than body temperature.

FIG. 4 depicts the composite stent of FIG. 3 wherein the assembly is in the contracted wound shape, as the first element, has been programmed for that shape at higher than body temperature at its corresponding Austenite phase.

FIG. 5 shows a hysteresis loop exhibited by superelasticity for both the composite stent of FIG. 1 and the composite stent of FIG. 3.

FIG. 6 shows the hysteresis relationship as respects phase transformation.

FIG. 7 shows a sample segment bent 90 degrees to show assembly flexibility.

FIG. 8 shows that the height of the peaks is approximately equal or slightly larger than the distance between the peaks.

FIG. 9 shows the preferred arrangement of the helically wound turns, wherein the peaks of one turn follow without touching the valleys of the adjacent turn at the start of the windings.

FIG. 10 shows the peaks of one turn in line with the peaks of the adjacent turn without touching at the start of the windings.

### DETAILED DESCRIPTION OF THE INVENTION INCLUDING PREFERRED EMBODIMENTS

The Figures which accompany this description make reference to the elements of the composite stent, which may be same in more than one Figure. Accordingly, such common element is assigned the same reference numeral even it appears in different Figures.

Composite Stent Assemblies

In FIG. 1, the composite stent is depicted as a composite structure, with a first element of the composite stent in the Austenite phase, at body temperature, bonded to a second element in the Martensite phase, at body temperature. In this FIG. 1, this composite/assembly is in the wound, contracted state. Each of the first and second element can occur as alternating linear segments in the composite stent assembly, or, alternatively, comprise concentric helical structures, with one structure surrounding the other, or alternatively, the first and second elements, can be concurrently wound about a common axis, with each element separated from its adjacent winding. Each of these adjacent windings are bonded to one another at regular intervals, thereby forming an integral composite stent assembly.

In practice the stent of FIG. 1, functions by first deployment within the lumen of vessel at body temperature, and, thereafter, is subjected to an elevated temperature within the body, by inductance heating, by circulating a heated saline solution, or by other suitable means, within the deployment hardware, such as a laser. For the purposes of clarity and focus, only inductance heating will be further described.

Both elements of the composite, when at room temperature in the Martensite phase, at the Martensite finish temperature (“M_{f}”), are soft and pliable. Each element is preferably of the same length when stretched in a straight line. Moreover, both elements may have essentially the same, and preferably, equal masses. When at room temperature, in the Martensite phase, each element of the composite can be bonded to the other to form a composite assembly. The sum of the masses of the two elements equates to essentially the same mass of a commonly used single element stent.

The first element of the composite stent is programmed, when manufactured, to have, at the Austenite finish temperature (“A_{f}”), a smaller length and pitch than the second element. The term “programmed” and “preprogrammed” are understood to include, within the context of this invention, the superelastic and memory characteristics of an element of a composite stent, based upon its composition and processing history. Thus, within the context of this invention, a programmed element of a stent, has predictive behavior, relative to its counterpart with a composite assembly; and, in relation to the ambient temperature in which it is used and/or deployed.

This first element, as noted above, is bonded to a second element, which is programmed when manufactured, to have, at the Austenite finish temperature (“A_{f}”), a length and pitch larger than the first element. Both elements are programmed, when manufactured, to have essentially the same outside diameter of the helix, when in their respective Austenite finish temperature (“A_{f}”).

The method for manufacturing stents is well-known and utilize readily available equipment and techniques, see for example, U.S. Pat. No. 5,421,955 (to Lau), which is herein incorporated by reference in its entirety. Moreover, it is well-known that stents can be readily manufactured from memory metals, such, as Nitinol, in accordance with such established techniques, see for example U.S. Pat. No. 5,514,154 (to Lau) and the references of record cited therein, all of which are herein incorporated by reference in their entirety.

Each of the elements of the composite stent are bonded together into a composite stent assembly, while each element is in the pliable, or Martensite phase, by well-known, commonly used methods and materials. For instance, a coaxial assembly can be formed with the first element, from a square or rectangular wire, inserted into a thin walled square or rectangular tube with the longer or flat side in constant contact with the wall of the lumen. The rectangular wire shape is preferred because it maximizes the area of contact with the lumen wall, and minimizes lumen interference. Other combinations may also be used, such as two square wires bonded together with a biocompatible plastic such as polyurethane; two wires of various cross-sections bonded together by suitable means such as welding, twisting; or, a mesh embodying the two elements to function in the spirit of this invention to longitudinally expand, contract and rotate the stent assembly.

The transformation temperatures, and the Hysteresis Loop of the alloys, from which these elements are formed, can be precisely controlled by well-known modeling techniques, and published data, relating to their extremely sensitive nickel-titanium ratio; and, by alloying additions such as oxygen nitrogen and additions of metals such as Co, V. Fe, Al, Pt, Ng and Cg. For instance, Hysteresis Loop width can range from 10 degrees centigrade, for certain nickel titanium copper, which allows for a much higher width for other alloys.

Stent Design Preferences

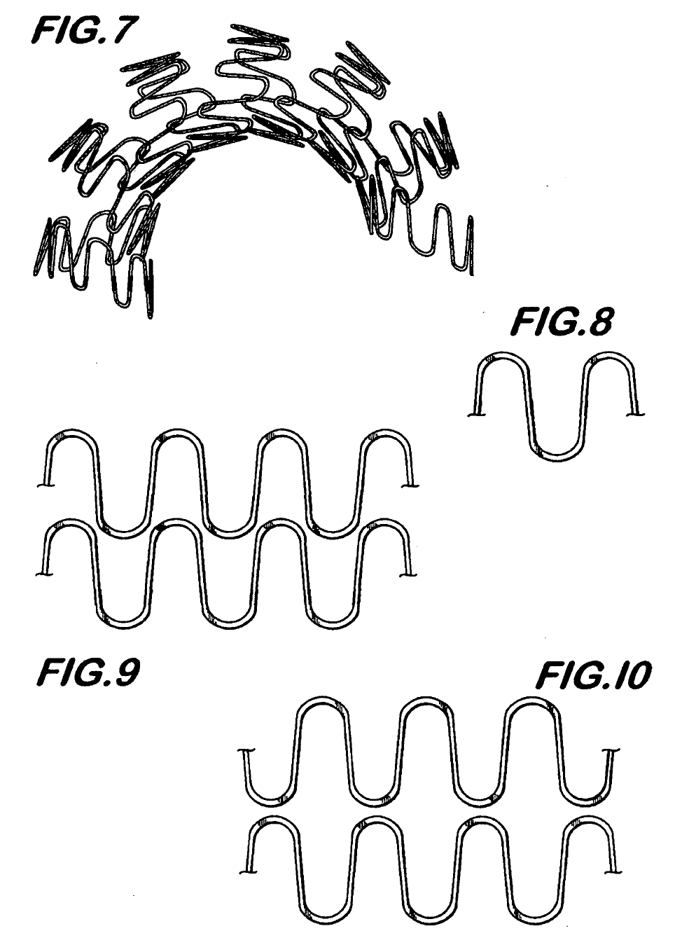

The helically wound turns are formed with numerous undulating curves or waves, as illustrated in FIG. 8, that function in close relationship with each other in order to produce maximum surfaces of contact in both longitudinal and rotational directions. For example, in the FIG. 9 each of the undulating wave-like concentric windings appear to mirror each adjacent winding. Conversely, FIG. 10 illustrates each of the peaks of each concentric windings are proximate to one another.

The preferred shape, as illustrated in the above Figures, is thus a helically wound assembly of the two elements bonded together, each turn having numerous curved peaks programmed in a shape that the curved peaks of one turn follow without touching the underside of the peaks or valleys of the other turn, both at the start and finish of the cycle of contraction, expansion and rotation of the assembly. The shapes of the peaks and valleys are programmed to remain constant to one another regardless the relative movement of each element in the unwinding, winding and rotation of the assembly.

The undulating or wavy design of the helically wound elements maximizes the unclogging and breaking up of the plaque with a steady, gentle sweeping, wiping action lengthwise and rotating wise along the lumen walls and at the same time can be highly resistant to crushing. Conversely, a balloon expanded stainless steel stent has to compensated for “spring back”, by increasing its outside diameter which may cause nicks and stretches in the walls of the lumen. For example, to fill a 5 mm lumen the stent might have to be expanded to 6 mm, potentially causing damage to the vessel itself. In contrast to stainless steel, the dual element Nitinol stent transforms directly to its preprogrammed shape with no spring back.

The helically wound shape of the composite stent also adapts more easily to any irregularities of the walls of the vessel. The pressure against the wall of the vessel can be designed to be very gentle to prevent damage to the endothelial cells of the vessel walls while keeping the stent in place. This very light continuous contact and gentle radial pressure on the vessel wall, is enhanced by the plurality of turns with multiple peaks and valleys of the stent. Thus, no outward large pressure is needed to unclog the plaque, as it is the case with a balloon inflated stainless steel stent. The superelastic Nitinol assembly of this invention, which is 20 times more flexible than stainless steel, is also easier to guide through tortuous paths.

The undulating design of the helically wound assembly also allows the stent to adjust to the shape of the vessel, and remain in place with a rninimal pressure on the vessel walls.

Stent Deployment Methods

The dual element assembly can be delivered to the desired site in the lumen by conventional catheterization.

This composite stent of FIG. 1, is placed, in its constrained configuration, inside a catheter at room temperature and guided to the site in the lumen where it is to be deployed from the catheter, at body temperature. The stent assembly can be prevented from premature deployment by chilling, or by mechanical constraint provided by the catheter.

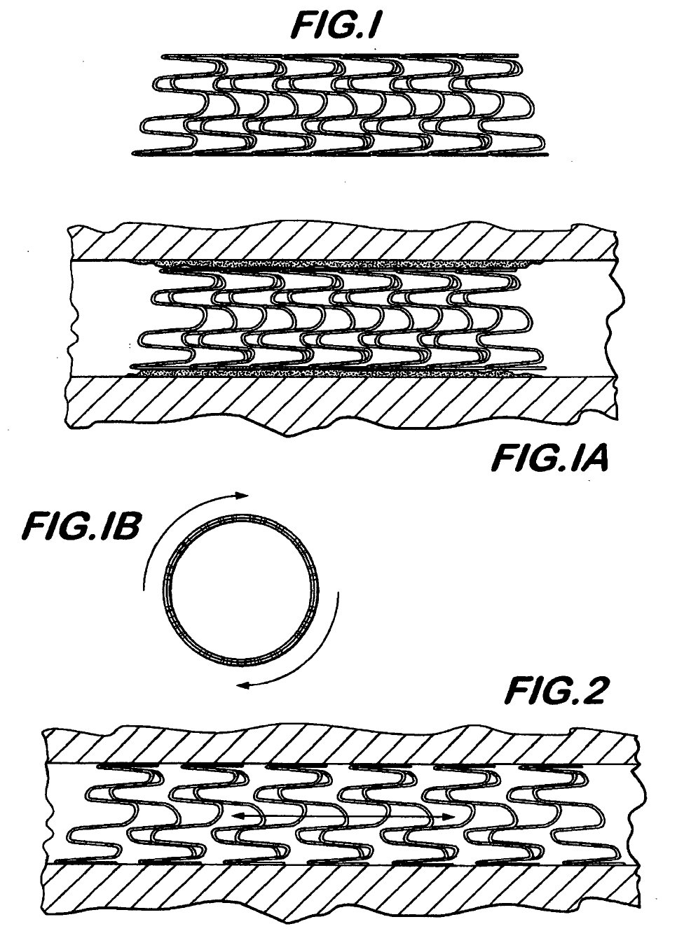

FIG. 2 depicts the transition of the stent of FIG. 1 from the constrained configuration to its fully deployed configuration. More specifically, the first element of the bonded assembly changes or transforms its pre-programmed shape from the Martensite phase to the Austenite phase at the Austenite finish temperature, thus, deploying the assembly in the shorter or contracted shape. The second element, which has remained soft and pliable in the Martensite phase, is now transformed to its pre-programmed longer shape by an induction heating system which concentrates a beam of electromagnetic flux on the stent. The induction systems is programmed to generate a pulse of enough intensity and frequency to induce an Austenite finish temperature, pre-programmed for the second element, to transform to its longer unwound and rotated position shape, as depicted in FIG. 2. To maintain a constant outside diameter of the helix, the longer element unwinds and expands simultaneously, by rotating to its pre-programmed shape. The force produced in the transformation is very large, and more than enough to pull the first element bonded to the second element, thus, expanding and rotating the combined stent. When the longitudinal expansion is completed, the induction flux is programmed to stop. The second element returns to the soft pliable stage or Martensite finish temperature, thus, winding and rotating the combination to the shorter pre-programmed length, and pitch of the first element, thus finishing the expansion, contraction and rotation cycle.

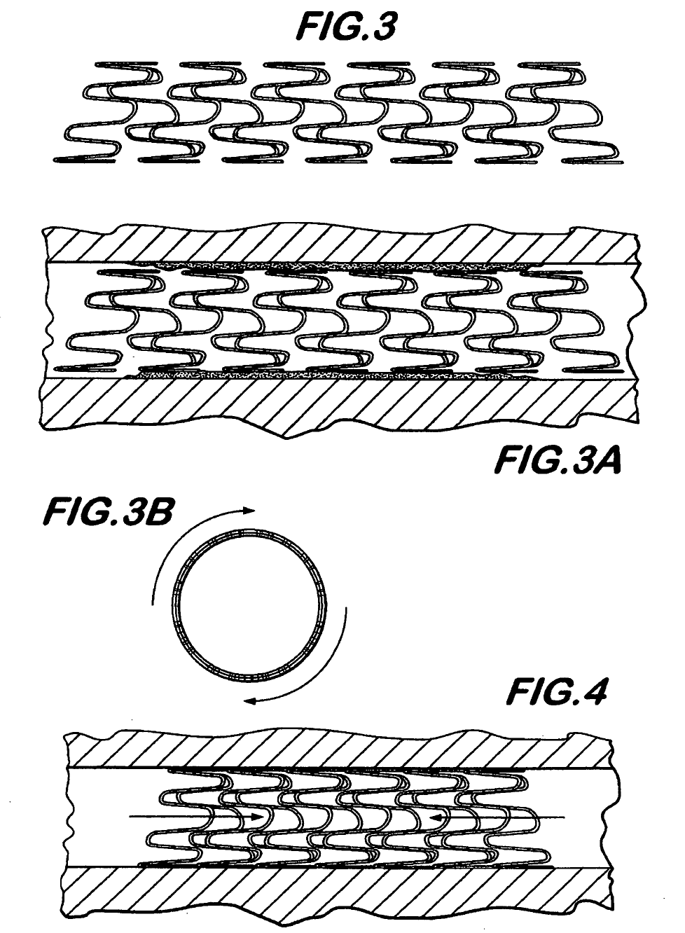

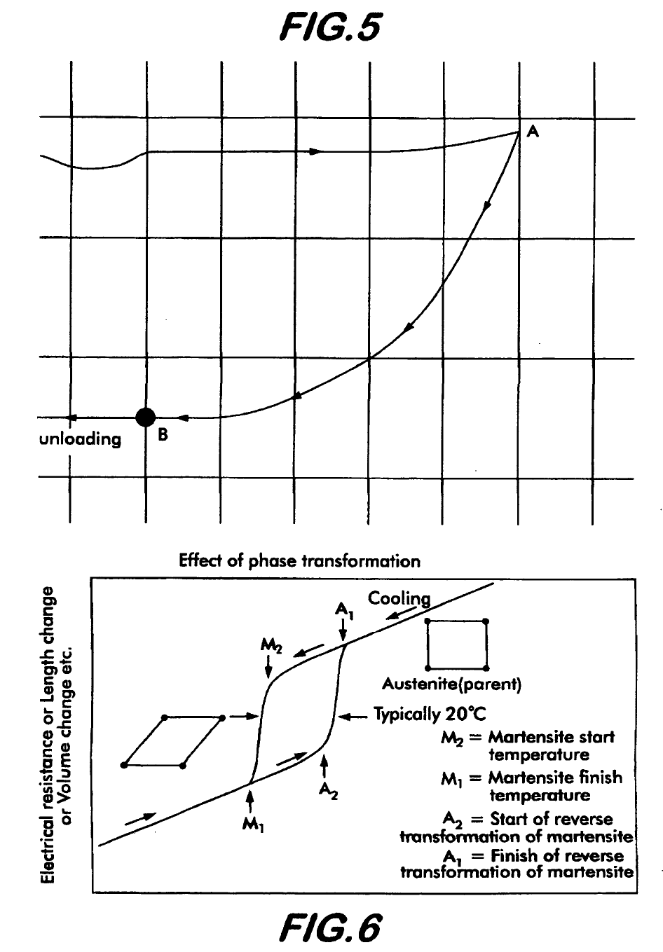

FIGS. 3 & 4 depict the inverse relationship of the first and second element of the composite stent of this invention. In this embodiment, the delivery and inductance heating procedures are the same as in the first option, but in this case the longer element (FIG. 3) is programmed to transform to the Austenite phase, at Austenite finish temperature, at the body temperature, and the shorter element (FIG. 4) is programmed to transform to the Austenite phase at an Austenite finish temperature higher than body temperature. When heating is stopped the stent assembly returns to the longer expanded shape (FIG. 3). Accordingly, the composite stents of this invention can be preprogrammed to follow either deployment cycle: (a) expansion, rotation, contraction, rotation; or (b) contraction, rotation, expansion, rotation. Consequently, it is the preprogrammed movement of the composite stent relative to the lumen wall, which creates a gentle wiping, sweeping action, without traumatizing the endothelial lining which surrounds the interior wall of the lumen. Because the action of the composite stent is preprogrammed to minimize trauma, the likelihood of clot formation from the displaced plaque is also minimized. In any event, it may be medically prudent to implement the deployment of the composite stent of this invention in conjunction with an embolic protection device, of the type disclosed in U.S. Pat. No. 6,702,834 (to Boylan), which is herein incorporated by reference in its entirety.

Unclogging of the lumen can be determined by conventional monitoring procedures such as angiograms followed up by intravascular ultrasound. Where repeated wiping sweeping of the occluded area is warranted, the inductance heating of the composite stent can be repeated. Upon satisfactory, displacement of plaque from the occluded area of the vessel lumen, the inductance heating system is removed and the stent assembly remains anchored at the deployment site inside the lumen. Depending upon the preprogrammed configuration of the composite stent, its final deployed configuration will either correspond to the composite stent of FIG. 1 or FIG. 3.

The composite stent is designed to remain secured inside the lumen with minimal gentle contact force made possible by the hysteresis characteristics of Nitinol, and by the shape of the helical turns with multiple peaks and valleys.

In one of the preferred embodiments of the method of this invention, a conventional monitoring system can be programmed to control the expansion, contraction and/or rotation cycles of the stent. More specifically, a preventive periodic schedule can be programmed, according to each patient's needs and medical experience, to reattach and reactivate the induction heating system. This periodic movement will also prevent scarring. If unclogging and breaking up of the plaque in small particles is accomplished in one or more cycles which may be programmed to rotate half a turn or more, the induction heating system can be removed leaving the stent assembly anchored in the shorter or contracted shape or in the expanded longer one.

Induction Heating Activation

The above described characteristics of this novel stent, coupled with conventional monitoring and a preventive periodic reactivation program, minimizes the need for repeated surgeries and angioplasties. Moreover, there is always the option to reattach and energize the induction heating system, if required. More specifically, in one of the preferred embodiments of this invention, the inductance heating system is composed of a spiral primary induction coil placed in front of the heart to produce an electromagnetic flux aimed at the dual stent, or stents, in order to induce a heating current in the corresponding element of the dual stent assembly.

The power and duration of this induction heating cycle can be modulated in accordance with well-known techniques and monitoring methods. More specifically, its is known that induction heating is a function of (a) frequency and amplitude of the activating applied alternating current; (b) the dimensional cross section, or the work, in this case the stent; (c) the linear length of the stent; (d) the shape of the stent; and (e) the electric and magnetic properties of the material from which the composite stent is manufactured.

Thus, it is possible with existing technology, to reach the required transition temperature, to effect the phase changes, within fractions of a second. The power to energize the primary inductance coil can be supplied by relatively high frequency alternating current, converted from direct current available from long life rechargeable lithium batteries or infolithium rechargeable batteries, that indicate how much power is left, or any other suitable batteries. Conversion of power, controls, programming and any other function required by the system can be accomplished by the use of a microchip in accordance with existing technology.

As the mass of the dual element stent is very small, the energy required to activate the system is also very small, therefore, all the components of the system can be contained in a lightweight patch or shoulder holsters attached to the skin in front of the heart.

As it is usual practice with any type of stent, care should be taken to select the size of the stent namely, the outside diameter and length. However, the stent subject of this invention is more adaptable to the shape of the vessel due to its novel design.

Computer visualization techniques provide cardiologists with the means to determine the correct size of the stent. This computer technology enables doctors to view test results, without going to the catheterization laboratory, and can be taken on a laptop to the patients bedside. The proportion of longitudinal expansion, and consequently rotation and the choice of either the first (FIG. 1) or second option (FIG. 3), is to be determined according to the needs of the patient.

What I claim is:

1. An assembly for implanting by catheterization within a lumen or the like in the body, comprising:
(a) an assembly of two elements made of shape memory superelastic material selected from the group consisting essentially of Nitinol, Flexon, Niti and any combination thereof;
(b) the two elements to be of the same length when elongated or stretched lineally;
(c) the two elements bonded by suitable means, including inserting a wire into a thin walled tube for a coaxial arrangement, preferably a rectangular wire into a rectangular thin walled tube, or two square wires bonded with a biocompatible material, such as polyurethane, in such a manner that the wider side of the assembly is in continuous contact with the vessel wall;
(d) the two elements programmed when manufactured to produce a longitudinal and rotating movement of the assembly, maintaining a constant outside diameter, said movements could be as small as to comprising half a turn unwinding plus half a turn winding, or vice versa per cycle;
(e) a plurality of helically wound turns with a plurality of peaks and valleys in each turn which coupled with the different paths of the superelasticity hysteresis loop when loading and unloading allows to control the pressure against the lumen wall to a minimum for a very gentle sweeping or wiping action on the lumen wall while simultaneously opposing the forces tending to close the lumen;
(t) the outside diameter of the assembly remains constant by compensating the effect of the longitudinal movement by unwinding while expanding longitudinally and winding while contracting longitudinally thus rotating the assembly counterclockwise while expanding longitudinally and rotating the assembly clockwise while contracting longitudinally;
(g) the peaks and valleys are programmed to retain the same shape regardless the movement of the assembly, the height of the peaks being approximately equal to the distance between the peaks;
(h) both points at the ends of the assembly are bent in to form a closed loop.

2. The assembly of claim 1, wherein preferably the peaks of a turn follow without touching the valleys of the adjacent turns.

3. The assembly of claim 1, wherein the peaks of one turn are in line without touching with the peaks of the adjacent turns.

4. The assembly of claim 1, wherein:
(a) the first element is programmed to be deployed in the shorter, wound and longitudinally contracted shape at an Austenite finish temperature A_{f} in the range of normal body temperature, with a smaller pitch and shorter length than the second element bonded to it;
(b) the second element bonded to the first element is programmed to remain in the Martensite phase until heat is applied;
(c) when heat is applied to the assembly and Austenite finish temperature AF2 is reached at higher than body temperature the shape of the second element is programmed to simultaneously unwind and rotate counterclockwise while longitudinally expanding and pulling the first element bonded to it which is possible by the large force of transformation of the second element and the superelasticity of the first element;
(d) when heating is stopped the second element returns to the Martensite phase allowing the first element to pull the assembly to the shorter position simultaneously winding and rotating the assembly clockwise, thus finishing one cycle;
(e) after the plaque is removed in small particles the system can be removed and the stent assembly can be left secured in the short, longitudinally contracted wound shape.

5. The assembly of claim I, wherein:
(a) the second element is programmed to be deployed in the longer, unwound and longitudinally expanded shape at an Austenite finish temperature A_{f} in the range of normal body temperature at A_{f} with a larger pitch and longer length than the first element bonded to it;
(b) the first element bonded to the second element is programmed to remain in the Martensite phase until heat is applied;
(c) when heat is applied to the assembly and Austenite finish temperature A_{f} is reached at higher than body temperature the shape of the first element is programmed to simultaneously wind and rotate clockwise while longitudinally contracting and pulling the second element bonded to it which is possible by the large force of transformation of the first element and the super elasticity of the second element;
(d) when heating is stopped the first element returns to the Martensite phase allowing the second element to pull the assembly to the longer position simultaneously unwinding and rotating the assembly counterclockwise thus finishing one cycle;
(e) after the plaque is removed in small particles the system can be removed and the stent assembly can be left secured in the long longitudinally expanded unwound shape.

6. The assembly of claim 5, wherein:
(a) the assembly is connected to a guide line made of a biocompatible, conducting, superelastic material such as Nitinol, and guided in its elongated shape to the segment in a vessel in the brain with a blood clot the assembly being at body temperature in its elongated shape when inserted, when heat is applied the assembly will transform winding while simultaneously contracting and rotating clockwise facilitating the removal of the blood clot by pulling out the guide line with the blood clot. Heating may be applied by inductance either from an outside source or self inductance produced by applying a relativity high frequency current through the line.

7. The assembly of claim 5, wherein:
(a) the size and shape of the assembly is modified to remove a blood clot in a case of deep vein thrombosis;
(b) inductance heating may be applied by suitable means.

8. The assemblies of claims 4 and 5, wherein the movements of the assembly may be synchronized with the heart beats in accordance with the cycles period which may be larger than the heart beat period.
